(19) United States
(12) Patent Application Publication
(10) Pub. No.: US 2004/0132060 A1
(43) Pub. Date: Jul. 8, 2004

(54) Variant of HNF-1alpha gene having novel single nucleotide polymorphism and a variant protein encoded by the same

(76) Inventors: Yeon-su Lee, Kyungki-do (KR); Sung-young Young Jeong, Kyungki-do (KR); Kyu-jeung Ahn, Seoul (KR); Byung-joon Kim, Seoul (KR); Sook-young Kim, Kyungki-do (KR); Mi-kyung Kim, Daejeon (KR)

Correspondence Address: CANTOR COLBURN, LLP, 55 GRIFFIN ROAD SOUTH, BLOOMFIELD, CT, 06002

(21) Appl. No.: 10/663,857

(22) Filed: Sep. 15, 2003

(30) Foreign Application Priority Data
Sep. 16, 2002 (KR) 2002-56229

(51) Int. Cl. C12Q 1/68; C07H 21/04

(52) U.S. Cl. 435/6; 536/23.2

(57) ABSTRACT

A polynucleotide or nucleic acid fragment comprising a new single nucleotide polymorphism of a human HNF-1α gene. The polynucleotide or nucleic acid fragment includes a polymorphic site having adenine (A) at position 1699 of SEQ ID NO: 1 or having thymine (T) at position 29 of SEQ ID NO: 3, and more than 10 contiguous nucleotides set forth in SEQ ID NO: 1 or 3.

This application claims the priority of Korean Patent Application No. 2002-56229, filed Sep. 16, 2002, in the Korean Intellectual Property Office, the disclosure of which is incorporated herein in its entirety by reference.

### BACKGROUND OF THE INVENTION

1. Field of the Invention

The present invention relates to a nucleic acid fragment comprising a polymorphic site of SEQ ID NO: 1 having adenine (A) nucleotide at position 1699, or a polymorphic site of SEQ ID NO: 3 having thymine (T) nucleotide at position 29, and comprising more than 10 contiguous nucleotides set forth in SEQ ID NO: 1 or 3, or a complement thereof, and a protein variant comprising the polymorphic site.

2. Description of the Related Art

Conventionally, various methods for analyzing a genetic variation have been widely used in the medical field to diagnose the occurrence of and/or propensity to a disease. The methods include a method comprising amplifying a gene by using an nucleic acid amplification method such as polymerase chain reaction (PCR), and analyzing the nucleotide sequence of the amplified gene by using nucleotide sequencing, hybridization or single strand conformational polymorphism (SSCP) analysis. For example, PCR and nucleotide sequencing are commercially used for BRCA gene probe. BRCA genes are known to be associated with breast cancer.

MODY3 genes give rise to maturity onset diabetes of the young, representing 10˜30% or higher of type 11 diabetes (Yamada et al., Diabetes 48:645, 1997; Yoshiuchi et al., Human mutation 18:345, 2001; U.S. Pat. No. 6,187,533; WO9811254).

To date, a few cases of MODY3 gene mutation have been reported in Korea (Kim et al., Korean Diabetes Association, 23:793, 1999), and the reported cases are not linked with new mutations.

Therefore, there is demand for studies of novel mutations for disease diagnosis, investigation of disease mechanisms and drug discovery. In particular, there is an increasing need to screen MODY3 gene mutations specific to racial and geographical characteristics.

### SUMMARY OF THE INVENTION

The present invention provides a nucleic acid fragment comprising more than 10 contiguous nucleotides having a new mutation site, and complements thereof.

The present invention also provides allele-specific oligonucleotides hybridizing to all or some of nucleic acids comprising the new mutation site.

The present invention provides a method of analyzing a nucleic acid, comprising determining a nucleotide sequence of the nucleic acids comprising the new mutation site.

Also, the present invention provides a variant or fragment of a human HNF-1α polypeptide having an amino acid sequence consisting of 10 or more amino acids having mutations of the amino acids corresponding to the nucleic acids comprising the new mutation site.

The present invention provides a method for analyzing a protein comprising determining the mutations of the amino acids corresponding to the nucleic acid sequence comprising the new mutation site.

### BRIEF DESCRIPTION OF THE DRAWINGS

FIG. 1 shows a genomic DNA having a portion containing a mutant nucleotide of HNF-1α according to the present invention; and

FIG. 2 shows a genomic DNA having intron 8 containing a mutant nucleotide of HNF-1α according to the present invention.

### DETAILED DESCRIPTION OF THE INVENTION

A nucleic acid fragment according to the present invention comprises a polymorphic site of SEQ ID NO: 1 having adenine (A) at position 1699, or a polymorphic site of SEQ ID NO: 3 having thymine (T) at position 29, and comprises more than 10 contiguous nucleotides set forth in SEQ ID NO: 1 or 3 or complements thereof.

In detail, the nucleic acid fragment according to the present invention may include any polynucleotide that contains a polymorphic site of SEQ ID NO: 1 having a nucleotide A at position 1699 and more than 10 contiguous nucleotides set forth in SEQ ID NO: 1. The nucleic acid fragment according to the present invention may also include a HNF-1α gene itself.

The nucleic acid fragment preferably includes 10 to 100 contiguous nucleotides, more preferably 10 to 50 contiguous nucleotides, and most preferably 10 to 20 contiguous nucleotides, set forth in SEQ ID NO: 1 or complements thereof. The polymorphic site may be positioned at any portion of the fragment.

Also, the nucleic acid fragment according to the present invention may include any polynucleotide that contains more than 10 contiguous nucleotides set forth in SEQ ID NO: 3, which corresponds to a sequence of intron 8 positioned between exon 8 and exon 9 of human HNF-1α.

The nucleic acid may be DNA, RNA or PNA, and may be in the form of either single- or double-stranded nucleic acid. The nucleic acid fragment may be either a natural one or a synthesized one.

In the present invention, the term “polymorphism” refers to the occurrence of two or more alternative genomic sequences or alleles in a genetically determined group. A “polymorphic marker or site” is the locus at which the variation occurs. Preferably, a marker has at least two alleles having the frequency of greater than 1%, more preferably 10% to 20%, in a selected group. The polymorphic site may be a single base pair.

Also, the present invention provides an allele-specific oligonucleotide comprising a polymorphic site of SEQ ID NO: 1 having an A nucleotide at position 1699 or a polymorphic site of SEQ ID 3 having a T nucleotide at position 29, and hybridizing to the nucleotide of SEQ ID NO: 3, or complements thereof.

In the present invention, the term “allele-specific” refers to specifically hybridizing to each allele. For example, hybridizing is performed so as to specifically identify SNP of SEQ ID NO: 1 having an A nucleotide at position 1699. The hybridization is performed under stringent conditions, for example, conditions of 1 M or less in salt concentration and 25° C. or higher in temperature. For example, conditions of 5×SSPE (750 mM NaCl, 50 mM Na Phosphate, 5 mM EDTA, pH 7.4) and a temperature of 25˜30° C. are suitable for allele-specific probe hybridization.

In the present invention, the allele-specific oligonucleotide may be a probe. The term “probe” used herein means a hybridization probe, that is, oligonucleotide capable of sequence-specifically binding with a complementary strand of a nucleic acid. Such a probe includes peptide nucleic acids as described in Science 254, 1497-1500 (1991) by Nielsen et al. The probe according to the present invention, which is an allele-specific probe, is hybridized to DNA fragments derived from one component but is not hybridized to DNA fragments derived from different components since there is a polymorphic site in nucleic acid fragments derived from two components of the same species. In this case, hybridization conditions are significantly different in hybridization stringency between alleles. Thus, the hybridization conditions should be stringent enough to allow hybridization to only one allele. The probe according to the present invention is preferably disposed such that the central site, that is, position 7 in the case of a 15 nucleotide probe, or position 8 or 9 in the case of a 16 nucleotide probe, is aligned with a polymorphic site of the sequence, causing a significant difference in hybridization between alleles. The probe according to the present invention can be used in diagnostic methods for detecting alleles. The diagnostic methods include detection methods based on a nucleic acid hybridization, e.g., southern blot and in case where DNA chips are used to detect a particular nucleic acid, the probe may be provided as an immobilized form on a substrate of a DNA chip.

In the present invention, the allele-specific oligonucleotide may be a primer. The term “primer” used herein refers to a single stranded oligonucleotide capable of acting as a starting point of template-directed DNA synthesis under appropriate conditions (for example, in a buffered conditions containing four different nucleotide triphosphates and polymerases such as DNA or RNA polymerase or reverse transcriptase) and at an appropriate temperature. The appropriate length of the primer may vary according to the purpose of use, generally 15 to 30 nucleotides. In general, a shorter primer molecule requires a lower temperature to form a stable hybrid with a template. A primer sequence is not necessarily completely complementary with a template but should be complementary enough to hybridize to a template. The primer is preferably arranged such that the 3′ end thereof is arranged with a polymorphic site of SEQ ID NO: 1 having a nucleotide A at position 1699 or a polymorphic site of SEQ ID NO: 3 having a nucleotide T at position 29. The primer hybridizes to a target DNA containing a polymorphic site, and start an allelic amplification in which the primer exhibits complete homology. The primer is used in pair with a second primer hybridizes at the opposite site. After amplification reaction, the amplified product is obtained from two primers, which means that there is a specific allelic form.

In accordance with another aspect of the present invention, there is provided a method of analyzing a nucleic acid comprising determining a nucleotide sequence at the polymorphic site of position 1699 of SEQ ID NO: 1 or of position 29 of SEQ ID NO: 3. The method may include isolating a nucleic acid such as gemonic DNA or RNA from a sample, and determining the sequence of the isolated nucleic acid and determining the nucleotide sequence at the polymorphic site of position 1699 of SEQ ID NO: 1 or of position 29 of SEQ ID NO: 3. If the analysis result shows that the nucleotide sequence at the polymorphic site of position 1699 of SEQ ID NO: 1 is a nucleotide other than guanine (G) or the nucleotide sequence at the polymorphic site of position 29 of SEQ ID NO: 3 is a nucleotide other than cytosine (C), it is determined that there is an increased risk of maturity onset diabetes of the young (MODY). Here, the nucleotide sequence is determined by general sequencing methods, for example, a dideoxy method (Sambrook et al., Molecular Cloning, A Laboratory Manual, 2^{nd} Ed. CSHP, New York 1989), or using automated apparatuses.

A variant or fragment of human HNF-1α polypeptide according to the present invention, comprises a polymorphic site at which an amino acid at position 567 of SEQ ID NO: 2 is an amino acid other than valine (Val), preferably, isoleucine (Ile), and comprises more than 10 contiguous amino acids derived from the amino acid sequence of SEQ ID NO: 2. As set forth in SEQ ID NO: 2, the variant or fragment of human HNF-1α polypeptide according to the present invention, is preferably derived from the amino acid sequence of SEQ ID NO: 2 having Ile, instead of Val, at position 567.

The protein analyzing method of the present invention comprises determining the amino acid sequence at position 567 of SEQ ID NO: 2. The amino acid sequence is determined by general methods, for example, N- or C-terminal identification or using an apparatus such as MS/MS, or MALDI-TOF.

The present invention will now be described in more detail by referring to the following examples. However, these examples are provided for illustration only and the present invention is not limited to those examples.

### EXAMPLES

Genomic DNA was isolated from blood samples of 97 people who are clinically presumed as MODY patients, 10 exon and intron portions of MODY3 genes were amplified for base sequencing and MODY3 gene variations were detected.

### Example 1

### Amplification of 10 Exon DNA in Human HNF-1α Gene

Blood was collected from 97 patients, and genomic DNA was isolated therefrom using a QIAGEN plasmid midi kit (QIAGEN; Germany), 10 MODY3 exons were amplified using a PCR reaction solution (Table 1). Conditions of PCR amplification were 5 minutes (95° C.) for initial denaturation, 30 cycles of 30 seconds (95° C.) for denaturation, 15 seconds (62° C.) for annealing and 30 seconds (72° C.) for 5 extension, and 3 minutes(72° C.) for final extension. Primers used in PCR amplification are listed in Table 2.

Among primers listed in Table 2, T7 promoter sequence (SEQ ID NO: 24) was added into forward primers, and T3 promoter sequence (SEQ ID NO: 25) was added into 5′ terminal of reverse primers.

TABLE 1
Composition of PCR reaction solution

| Ingredient | Volume (μl) |
|---|---|
| DNase, RNase-free water | 12.8 |
| dNTP mix (2.5 mM/nucleotide) | 2 |
| 10x Taq polymerase buffer | 2 |
| Primer set (10 pmol/primer) | 2 |
| Gemonic DNA (100 ˜ 1.0 μg) | 1 |
| Taq polymerase (5 units/μl) | 0.2 |

TABLE 2
PCR primers used in amplification of MODY3 gene

| | Sequence |
|---|---|
| Mody 3 promoter sense(T7) | SEQ ID NO: 4 |
| Mody 3 promoter antisense(T3) | SEQ ID NO: 5 |
| Mody 3 exon1 sense(T7) | SEQ ID NO: 6 |
| Mody 3 exon1 antisense(T3) | SEQ ID NO: 7 |
| Mody 3 exon2 sense(T7) | SEQ ID NO: 8 |
| Mody 3 exon2 antisense(T3) | SEQ ID NO: 9 |
| Mody 3 exon3 sense(T7) | SEQ ID NO: 10 |
| Mody 3 exon3 antisense(T3) | SEQ ID NO: 11 |
| Mody 3 exon4 sense(T7) | SEQ ID NO: 12 |
| Mody 3 exon4 antisense(T3) | SEQ ID NO: 13 |
| Mody 3 exon5 sense(T7) | SEQ ID NO: 14 |
| Mody 3 exon5 antisense(T3) | SEQ ID NO: 15 |
| Mody 3 exon6 sense(T7) | SEQ ID NO: 16 |
| Mody 3 exon6 antisense(T3) | SEQ ID NO: 17 |
| Mody 3 exon7 sense(T7) | SEQ ID NO: 18 |
| Mody 3 exon7 antisense(T3) | SEQ ID NO: 19 |
| Mody 3 exon8 & 9 sense(T7) | SEQ ID NO: 20 |
| Mody 3 exon8 & 9 antisense(T3) | SEQ ID NO: 21 |
| Mody 3 exon10 sense(T7) | SEQ ID NO: 22 |
| Mody 3 exon10 antisense(T3) | SEQ ID NO: 23 |

Amplified products were purified using a QIAquick kit. The purified products were used as templates for nucleotide sequence analysis.

### Example 2

### Nucleotide Sequence Analysis of MODY3 Gene

Nucleotide sequencing PCR was carried out using each purified primer for 10 MODY3 Exons by an ABI PRISM BigDye terminator cycle sequencing ready reaction kit (Applied Biosystem, U.S.A.) method, and the products were subjected to alcohol precipitation, followed by suspending in formamide, boiling at 5 minutes at 95° C., immediately dipping into 4° C. ice. Finally, sequence analysis was performed using an ABI PRISM 3700 Genetic Analyzer (Applied Biosystem, U.S.A.).

### Example 3

### Analysis for Detecting Mutations of MODY3 Genes

DNA sequences resulting from the sequence analysis were compared with nucleotide sequences in NCBI database, and genetic mutations were analyzed using a DNAstar program (DNASTAR, Inc, U.S.A.).

10 exon sequences of MODY3 genes, which were amplified from a DNA sample of 97 patients diagnosed with type II diabetes, were used for analysis.

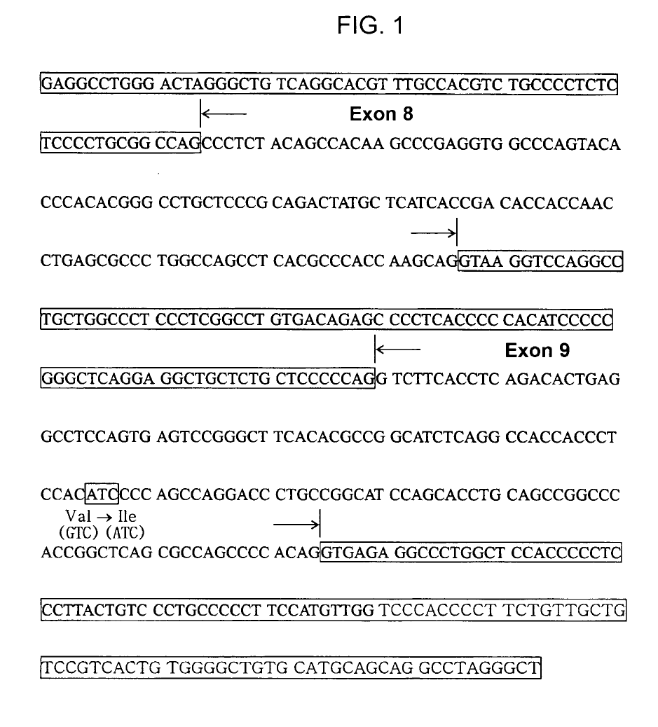

The result showed that mutations were found in 4 patients and a new mutation was found in one of the 4 patients. In other words, it was found that G at position 1699 of exon 9 in MODY3 gene of the patient was altered to A (FIG. 1). Also, it could be suggested that valine (Val) at position 567 in the amino acid sequence encoded by MODY3 gene was altered to isoleucine (Ile) (SEQ ID NO: 2).

### Example 4

### Nucleotide Sequence Analysis of Intron in MODY3 Gene

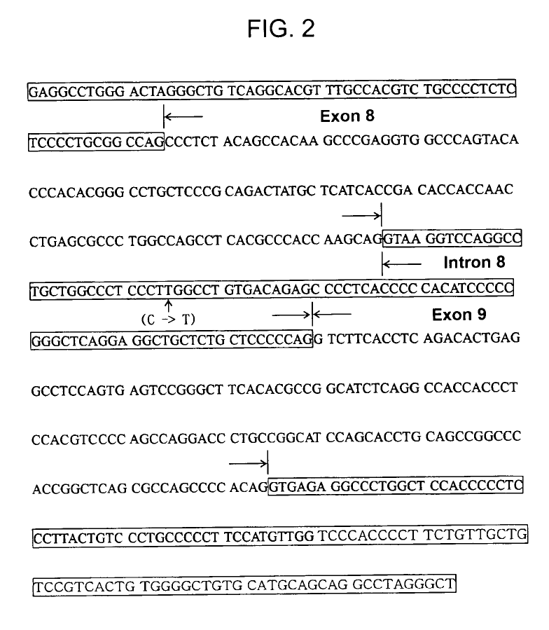

The same methods shown in Examples 1 through 3 were used for nucleotide sequence analysis of introns in HNF-1α. The analysis result showed that a new mutation was found in one patient. That is, C at position 29 of intron 8 positioned between exon 8 and exon 9 of HNF-1α was altered to T (FIG. 2).

Maturity onset diabetes of the young (MODY) cases among diabetic patients are relatively lower in Korea than in Europe in which MODY accounts for 10% of all diabetes cases, which is presumably because distributions of MODY genes are different depending on racial and geographical characteristics. Thus, further studies on MODY distributions must be conducted.

The nucleic acid fragment according to the present invention can be effectively used as a probe or primer in diagnosis of MODY.

The allele-specific oligonucleotide according to the present invention can be used as a probe or primer in diagnosis of MODY.

The variant or fragment human HNF-1α polypeptide according to the present invention can be effectively used as a probe or primer in diagnosis of MODY.

Also, the protein analyzing method according to the present invention can be effectively used as a probe or primer in diagnosis of MODY through peptide sequence analysis.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A nucleic acid fragment comprising a polymorphic site of SEQ ID NO: 1 having adenine (A) at position 1699, or a polymorphic site of SEQ ID NO: 3 having thymine (T) at position 29, and comprising more than 10 contiguous nucleotides derived from nucleotide sequence set forth in SEQ ID NO: 1 or 3, or a complement thereof.

2. The nucleic acid fragment of claim 1, wherein the nucleic acid fragment comprises 10 to 100 contiguous nucleotides, or a complement thereof.

3. An allele-specific oligonucleotide hybridizing to the nucleic acid fragment of claim 1 or a complement thereof.

4. The allele-specific oligonucleotide of claim 3, wherein the oligonucleotide is a probe.

5. The allele-specific oligonucleotide of claim 3, wherein the oligonucleotide is a primer.

6. The allele-specific oligonucleotide of claim 5, wherein the 3′ end of the primer is arranged with the polymorphic site of the nucleic acid fragment.

7. A method for analysing a nucleic acid comprising determining a nucleotide sequence of the polymorphic site at position 1699 of SEQ ID NO: 1 or at position 29 of SEQ ID NO: 3.

8. The method of claim 7, wherein if the nucleotide sequence of the polymorphic site at position 1699 is A or the nucleotide sequence of the polymorphic site at position 29 is T, it is determined that there is an increased risk for maturity onset of diabetes of the young (MODY).

9. A variant or fragment of human HNF-1α polypeptide, comprising a polymorphic site of an amino acid at position 567 of SEQ ID NO: 2, and comprising more than 10 contiguous amino acids derived from the amino acid sequence of SEQ ID NO: 2.

10. The variant or fragment of HNF-1α polypeptide of claim 9, wherein the amino acid at position 567 is isoleucine.

11. A method for analyzing a protein comprising determining the amino acid sequence at a position 567 of SEQ ID NO: 2.

12. The method for analyzing a protein of claim 11, wherein if the amino acid at position 567 is isoleucine, it is determined that there is an increased risk for maturity onset diabetes of the young (MODY).


SEQUENCE LISTING (25 sequences)

SEQ ID NO: 1 - DNA, 1896 bp, Homo sapiens
  CDS (1)..(1893): amino acid sequence of HNF-1a
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagaaccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacatccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

SEQ ID NO: 2 - protein, 631 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQNPFMATMAQLQSPHALYSHKPEVAQYTHTGLLPQTMLITDTTNLSALASLTPTKQVFTSDTEASSESGLHTPASQATTLHIPSQDPAGIQHLQPAHRLSASPTVSSSSLVLYQSSDSSNGQSHLLPSNHSVIETFISTQMASSSQ

SEQ ID NO: 3 - DNA, 93 bp, Homo sapiens
gtaaggtccaggcctgctggccctcccttggcctgtgacagagcccctcacccccacatcccccgggctcaggaggctgctctgctcccccag

SEQ ID NO: 4 - DNA, 41 bp, Artificial Sequence
  sense primer for amplifying promoter of MODY3 gene
taatacgactcactatagggtggccgtgagcatcctctgcc

SEQ ID NO: 5 - DNA, 39 bp, Artificial Sequence
  antisense primer for amplifying promoter of MODY3 gene
gtaaccctcactaaagggacgtgggttgcgtttgcctgc

SEQ ID NO: 6 - DNA, 40 bp, Artificial Sequence
  sense primer for amplifying exon 1 of MODY3 gene
taatacgactcactatagggcgtggccctgtggcagccga

SEQ ID NO: 7 - DNA, 40 bp, Artificial Sequence
  antisense primer for amplifying exon 1 of MODY3 gene
gtaaccctcactaaagggagggctcgttaggagctgaggg

SEQ ID NO: 8 - DNA, 42 bp, Artificial Sequence
  sense primer for amplifying exon 2 of MODY3 gene
taatacgactcactatagggcccttgctgagcagatcccgtc

SEQ ID NO: 9 - DNA, 40 bp, Artificial Sequence
  antisense primer for amplifying exon 2 of MODY3 gene
gtaaccctcactaaagggagggatggtgaagcttccagcc

SEQ ID NO: 10 - DNA, 40 bp, Artificial Sequence
  sense primer for amplifying exon 3 of MODY3 gene
taatacgactcactataggggcaaggtcaggggaatggac

SEQ ID NO: 11 - DNA, 42 bp, Artificial Sequence
  antisense primer for amplifying exon 3 of MODY3 gene
gtaaccctcactaaagggacgccgttgtacctattgcactcc

SEQ ID NO: 12 - DNA, 43 bp, Artificial Sequence
  sense primer for amplifying exon 4 of MODY3 gene
taatacgactcactatagggggctcatgggtggctatttctgc

SEQ ID NO: 13 - DNA, 42 bp, Artificial Sequence
  antisense primer for amplifying exon 4 of MODY3 gene
gtaaccctcactaaagggacgtgtcccttgtccccacatacc

SEQ ID NO: 14 - DNA, 42 bp, Artificial Sequence
  sense primer for amplifying exon 5 of MODY3 gene
taatacgactcactatagggtgctgaggcaggacactgcttc

SEQ ID NO: 15 - DNA, 42 bp, Artificial Sequence
  antisense primer for amplifying exon 5 of MODY3 gene
gtaaccctcactaaagggatacaagcaaggacactcaccagc

SEQ ID NO: 16 - DNA, 41 bp, Artificial Sequence
  sense primer for amplifying exon 6 of MODY3 gene
taatacgactcactatagggcccggacacagcttggcttcc

SEQ ID NO: 17 - DNA, 42 bp, Artificial Sequence
  antisense primer for amplifying exon 6 of MODY3 gene
gtaaccctcactaaagggaatccccaccagcttaccgatgac

SEQ ID NO: 18 - DNA, 40 bp, Artificial Sequence
  sense primer for amplifying exon 7 of MODY3 gene
taatacgactcactatagggcaggcctggcctccacgcag

SEQ ID NO: 19 - DNA, 40 bp, Artificial Sequence
  antisense primer for amplifying exon 7 of MODY3 gene
gtaaccctcactaaagggaggggctctgcagctgagccat

SEQ ID NO: 20 - DNA, 41 bp, Artificial Sequence
  sense primer for amplifying exon 8 and 9 of MODY3 gene
taatacgactcactatagggggcccagtacacccacacggg

SEQ ID NO: 21 - DNA, 40 bp, Artificial Sequence
  antisense primer for amplifying exon 8 and 9 of MODY3 gene
gtaaccctcactaaagggagggcagggacagtaagggagg

SEQ ID NO: 22 - DNA, 41 bp, Artificial Sequence
  sense primer for amplifying exon 10 of MODY3 gene
taatacgactcactataggggccttgtttgcctctgcagtg

SEQ ID NO: 23 - DNA, 41 bp, Artificial Sequence
  antisense primer for amplifying exon 10 of MODY3 gene
gtaaccctcactaaagggaggccatctgggtggagatgaag

SEQ ID NO: 24 - DNA, 20 bp, Artificial Sequence
  T7 promoter sequence
taatacgactcactataggg

SEQ ID NO: 25 - DNA, 19 bp, Artificial Sequence
  T3 promoter sequence
gtaaccctcactaaaggga